(19) United States
(12) Patent Application Publication
(10) Pub. No.: US 2009/0212238 A1
(43) Pub. Date: Aug. 27, 2009

(54) Apparatus for ion nitriding an aluminum alloy part and process employing such apparatus

(76) Inventors: Frederic Guernalec, Litre (FR); Denis Busardo, Gonneville/Mer (FR)

Correspondence Address: HEDMAN & COSTIGAN P.C., 1185 AVENUE OF THE AMERICAS, NEW YORK, NY, 10036, US

(21) Appl. No.: 10/587,465

(22) PCT Filed: Feb. 2, 2005
(86) PCT No.: PCT/FR05/00224

§ 371 Date: Sep. 22, 2006

(30) Foreign Application Priority Data
Feb. 4, 2004 (FR) 0401047
Feb. 21, 2004 (FR) 0401749
Jan. 31, 2005 (FR) 0500963

(51) Int. Cl. A61N 5/00

(52) U.S. Cl. 250/492.3

(57) ABSTRACT

The invention relates to a device for implanting ions in an aluminium alloy part (5), said device comprising an ion source (6) supplying ions accelerated by an extraction voltage, and first means for regulating (7-11) an initial beam (f1′) of ions emitted by said source (6) to form an implantation beam (f1). The source (6) is an electronic cyclotronic resonance source generating the initial beam (f1′) of multi-energy ions that are implanted in the part (5) at a temperature below 120° C. The implantation of said multi-energy ions of the implantation beam (f1) regulated by the regulating means (7-11) is simultaneously carried out at a depth controlled by the extraction voltage of the source.

### FIELD OF THE INVENTION

The subject matter of the invention is an apparatus for ion nitriding an aluminum alloy part by means of a nitrogen ion beam emitted by an ion source. The invention is also directed to a process for nitriding an aluminum alloy part employing such an apparatus.

The invention finds application for example in the field of plastics technology, where it is necessary to treat aluminum alloy parts that are used as mass production molds in the production of plastic parts.

### PRIOR ART

In the field of plastics technology, most plastic parts are manufactured by being molded in metal molds. Most of these molds are currently made of steel. This is because steel is a durable material that has good mechanical strength over time. Each steel mold can therefore be used to make a large number of plastic parts, on the order of 500,000 to 1,000,000 units. However, steel is a difficult material to treat and therefore is not conducive to getting products out on the market quickly. Nor does it allow for great flexibility of shape, whereas the current trend is to frequently change the shapes of plastic parts and thus the shapes of injection molds. For these reasons, the machining cost and time cost of a steel mold are relatively high.

There has consequently been a growing effort in the field of plastics technology to make injection molds out of a metal other than steel. Aluminum alloys constitute one of these metals. Aluminum alloy offers the advantage of having excellent machinability, that is, of permitting high-speed machining. Aluminum alloy also has a high heat exchange capacity, resulting in more rapid cooling of the plastic part, as well as being very lightweight and therefore easier to handle. The cost of a given volume of aluminum alloy is substantially comparable to that of an equal volume of steel.

A general problem that must be solved in this field is that aluminum alloy molds have limited mechanical strength over time, resulting in a low production capacity compared to those made of steel. The number of plastic parts produced in an aluminum alloy mold is typically on the order of 1,000 units. In addition, a specific problem to be solved in the field of aluminum alloy molds is that phenomena such as erosion of the molding surface, dulling of the joint plane, or corrosion develop more rapidly than they do in steel molds.

Manufacturers of aluminum alloy injection molds have been attempting to solve these problems by improving the surface mechanical strength of such molds. To do this, they have sought to enhance wear resistance by increasing surface hardness and lubrication (decreasing the friction coefficient) and by strengthening resistance to corrosion, which is due essentially to attack by chlorine compounds.

The prior art includes various chemical or physicochemical processes designed to improve the mechanical strength of aluminum alloy molds.

One such known chemical process is to anodize the aluminum alloy mold. Anodizing is an electrolytic process by which the natural layer of alumina (Al₂O₃) can be thickened to thicknesses on the order of 20 microns. This alumina layer is hard but very brittle (having substantially the same toughness as glass). In addition, it has a high thermal expansion coefficient and is sensitive to chlorine compounds, making it highly susceptible to thermal fatigue and corrosion.

Another chemical process is hard chroming. This is the electrolytic treatment of aluminum alloy molds to harden them. However, this process entails problems of uniformity of thickness at the edges of the molds. In addition, it requires a surface preparation known as pickling (the creation of 7- to 8-micron priming microroughnesses) whose quality depends on the knowhow of the subcontractor and which therefore has a poor reputation among mold-makers.

Another chemical process is nickel plating. This process consists in uniformly depositing a layer of Teflon-impregnated nickel in order to lubricate the surface. However, to impregnate nickel with Teflon the mold has to be maintained for several hours at a temperature of 250° C., which is fatal to the mechanical properties of aluminum alloys. Without Teflon, i.e., without lubrication, the nickel layer in turn is susceptible to the risk of delamination.

Another chemical process is vapor phase chromium nitride deposition. This method presents a problem with regard to the adhesion of the chromium nitride layer, which is of poor quality due to the low permissible temperature of application (beyond which the mechanical properties of the substrate are destroyed).

One physicochemical process is thermal nitriding. This consists in case-hardening a metal part with nitrogen to produce a high surface hardness. Such nitriding is generally performed thermally, that is, the metal part to be treated is heated to a temperature above 500° C. in a stream of ammonia gas. At that temperature, the ammonia gas dissolves and diff-uses into the alloy to form nitrides. See, for example, the document U.S. Pat. No. 4,597,808 (Arai Tohru et al.), which describes a physicochemical process of the above type. There is another problem, however, related to the type of materials to be treated, i.e. aluminum alloys. Such alloys contain hardening precipitates, which are obtained by temperings performed at 120 to 150° C. and which contribute to the good mechanical strength of these alloys. The problem is that raising the aluminum alloy to a temperature above 500° C., that recommended by U.S. Pat. No. 4,597,808, tends to remove these precipitates. It follows that the process described by U.S. Pat. No. 4,597,808 is unsatisfactory in terms of the desired mechanical strength of aluminum alloys.

There are other nitriding processes for aluminum parts intended for use in the electronics industry. The aim of these processes is to surface-treat the aluminum so as to deposit a fine layer of aluminum oxide or nitride that has attractive characteristics from an electronics standpoint, particularly good sound-insulating and heat-conducting characteristics, in order to preserve the electronic properties of the aluminum part. Examples can be found in the documents EP 1,288,329 (CCR GmbH Beschichtungs-techno) and U.S. Pat. No. 4,698,233 (Iwaki Masaya et al.), which describe such treatment processes for aluminum parts used in the electronics industry.

Elsewhere, the document U.S. Pat. No. 5,925,886 (Togiguchi Katsumi et al.) raised the possibility of producing an ion beam from an electron cyclotron resonance ion source (ECR source). It will be recalled that an ECR source has two main characteristics:
a magnetic field that confines the ions in a bounded volume located inside the source and known as the plasma chamber, and
a high-frequency wave released inside the source and designed to heat the electrons, which can then be ionized.

The chamber of the source contains a hot plasma composed of a mixture of magnetically confined ions and electrons. The ions can be exacted from the chamber through an opening and then accelerated. To produce gaseous ions (oxygen, nitrogen, neon, etc.), the chosen gas is introduced into the source in sufficient quantity to bring the ion beam to the required intensity.

### DESCRIPTION OF THE INVENTION

The object of the invention is to remedy the drawbacks and problems of the techniques described above.

The invention is directed in particular to an apparatus for implanting ions, particularly nitrogen ions, in an aluminum alloy part in order to improve the mechanical strength of said part.

The present invention is further directed to such an apparatus that is operative to treat the aluminum alloy in depth, typically over a thickness on the order of 0 to 3 μm, and the use of which does not cause a change in the mechanical characteristics of the part to be treated, permitting its use after treatment without reworking of the part.

The present invention is also directed to such an apparatus operative to treat specific areas of the aluminum alloy part.

The present invention is also directed to such an apparatus that does not require long treatment times.

Finally, the present invention is directed to such an apparatus that is inexpensive so that it can be used in an industrial context, the idea being that its cost should not being prohibitive in comparison to that of other treatment methods.

The inventive step of the present invention is to propose treating an aluminum alloy part by simultaneously implanting multi-energy ions at low temperature, more precisely at a temperature below 120° C. The ions are obtained by extracting, at one and the same extraction voltage, mono- and multi-charged ions created in the plasma chamber of an electron cyclotron resonance ion source (ECR source). Every ion produced by said source has an energy that is proportional to its charge state. It follows that the ions having the highest charge state, and thus the highest energy, come to be implanted in the alloy part at greater depths.

It will be noted at this stage of the description that this implantation procedure is fast and inexpensive, since it does not require a high extraction voltage from the ion source. To increase the implantation energy of an ion, it is economically preferable to increase its charge state rather than its extraction voltage.

It will also be noted that this apparatus makes it possible to treat a part without altering its mechanical properties, due to the presence of hardening precipitates obtained in advance via temperings performed at a temperature of between 120° C. and 150° C.

The apparatus for implanting ions in an aluminum alloy part comprises a source delivering ions accelerated by an extraction voltage and first means of adjusting an initial ion beam emitted by said source as an implantation beam.

According to the present invention, such an apparatus is chiefly recognizable in that said source is an electron cyclotron resonance source producing multi-energy ions that are implanted in the part at a temperature below 120° C., the implantation of the ions from the implantation beam being effected simultaneously at a depth controlled by the extraction voltage of the source.

More particularly, the process of the invention proposes using multi-energy nitrogen ions produced by the ECR ion source, into which the nitrogen has been introduced beforehand, and implanting the produced ions simultaneously in the aluminum alloy part, thereby creating aluminum nitride microcrystals which in turn cause an increase in hardness. The simultaneous implantation of these nitrogen ions can be performed at varying depths, depending on the requirements and the shape of the part. These depths depend on the implantation energies of the ions from the implantation beam, and can range from 0 to about 3 μm.

Due to a spray effect that differs with the energy and thus the charge state of the incident ion, the implanted ion concentration profile obtained is not the same, depending on whether for example N⁺, N₂⁺, N₃⁺ are implanted simultaneously, N⁺, N₂⁺, then N₃⁺ are implanted successively in increasing order of charge state, or N₃⁺, N₂⁺, then N⁺ are implanted successively in decreasing order of charge state. Successive implantation in increasing order of charge state results in a profile of broad thickness but low concentration. Successive implantation in decreasing order of charge state results in a profile of narrow thickness but high concentration. Simultaneous implantation is a compromise between the two preceding types of implantation that yields a profile of medium thickness and medium concentration. It is costly in terms of time to implant ions successively in increasing or decreasing order. The process of the invention recommends simultaneously implanting multi-energy ions with a multi-energy beam and is therefore both technically advantageous and ideal in terms of the physical compromise obtained (balanced concentration profile).

The increase in the hardness of the aluminum is related to the implanted nitrogen ion concentration. For example, with 10% implanted ions, the hardness of the part is increased locally by a ratio of 200%. In the case of aluminum, a 200% increase in hardness roughly corresponds to an intermediate hardness between that of titanium and that of steel. At 20% implanted nitrogen ions in the part, the hardness of the part increases by a ratio of 300%. In the case of aluminum, a 300% increase in hardness corresponds to a hardness equal or even superior to that of steel.

The process of the invention has one very attractive advantage over implantation performed with a mono-energy nitrogen ion beam: assuming that the implanted ion concentration is the same, an additional increase in hardness is observed with a multi-energy nitrogen ion beam. With an implanted ion concentration of 25%, a 60% increase in hardness has been measured in favor of implantation with a multi-energy beam over implantation with a mono-energy beam. Simultaneously implanting multi-energy ions brings about more effective mixing, due to collisions and cascades, of the various aluminum nitride layers (which are staggered at different implantation depths within the thickness being treated). The effectiveness of the processes of fragmentation and dispersion of the microcrystals constituting the aluminum nitride layers is undoubtedly the reason for this additional increase in hardness obtained by implantation with a multi-energy nitrogen ion beam. Multi-energy beams are particularly suitable for mechanical applications, whereas mono-energy beams are more specifically suitable for electronics applications, in which the creation of defects by collisions and cascades tends to degrade the electrical properties of the aluminum nitride (particularly its very high electrical resistance).

In application to aluminum alloy injection molds, the process of the invention makes it possible to obtain molds with a surface hardness close to that of steel, while still preserving the bulk mechanical properties of the aluminum alloy. The process of the invention also makes it possible to improve the anticorrosion characteristic of these aluminum alloy molds. Thus, the production capacity of an aluminum alloy mold treated with the simultaneous ion implantation nitriding process of the invention is greatly increased over that of a conventional aluminum alloy mold.

The apparatus of the present invention further advantageously comprises second means of adjusting the relative positions of the part and the ion source. It will be understood that relative displacement between the ion source and the part is effected so that the latter can be treated region by region. Thus, several regions of a single metal part can be treated to obtain identical or different hardnesses. The choice of the regions to be treated and the duration of the treatment they are to be given is governed by their functional specificity (for example, the region of the joint plane of the mold, the region of the molding surface).

According to a preferred embodiment of the apparatus of the present invention in which the part is movable with respect to the source, the two adjusting means advantageously comprise a part holder that is movable so that the part can be displaced as it is being treated. In another, non-preferred embodiment of the apparatus, it is the ion source that is displaced with respect to the part to be treated; this latter embodiment can be used when the part to be treated is very bulky.

The part holder is preferably equipped with cooling means to evacuate the heat generated in the part during the implantation of the multi-energy ions.

The first means of adjusting the ion beam accessorily comprise a mass spectrometer for sorting the ions produced by the source according to their charge and mass.

The first means of adjusting the initial ion beam preferably further comprise optical focusing means, a profiler, a current transformer and a shutter.

The apparatus is advantageously confined in an enclosure equipped with a vacuum pump.

The second means of adjusting the relative positions of the part and the ion source advantageously comprise means of calculating these positions from data related to the nature of the ion beam, the geometry of the part, the rate of displacement of the part holder with respect to the source, and the number of passes already completed.

According to a first variant of the process for treating an aluminum alloy by ion implantation using an apparatus according to the present invention, this process is chiefly recognizable in that the multi-energy ion beam displaces relatively with respect to the part at a constant rate.

According to a second variant of the process for treating an aluminum alloy by ion implantation using an apparatus according to the present invention, this process is chiefly recognizable in that the multi-energy ion beam displaces relatively with respect to the part at a variable rate that takes into account the angle of incidence of the multi-energy ion beam with respect to the surface of the part.

Regardless of whether it is the part to be treated or the ion source that is displaced, the relative rate of displacement between these two elements can be constant or it can be variable as a function of the angle of incidence of the beam with respect to the surface, at least for the duration of treatment of the region of the part. The rate can be managed differently for each to-be-treated region of the part. The rate depends on the beam emission rate, the concentration profile of the implanted ions and the number of passes. The rate can vary according to the angle of incidence of the beam with respect to the surface, in order to compensate for a small implantation depth by increasing the number of ions.

The multi-energy ion beam is preferably emitted at an emission rate and emission energies that either are constant or are variable and controlled by the ion source. As explained above, the process of the invention makes it possible to adjust the penetration depths of the multi-energy ions into the part. These penetration depths, which are staggered within the treated thickness, vary according to the different entrance energies of the ions at the surface of the part. More precisely, the ion source delivers ions with variable emission energies; in this case, the ion source is slaved so that the energies of the incident ions can be varied by manipulating the extraction voltage during each treatment.

Implanting nitrogen ions in the crystal structure of the part to be treated has the effect of creating extremely hard aluminum nitride microcrystals (having a face-centered cubic structure at low nitrogen concentrations and a compact hexagonal structure at high nitrogen concentrations) that lock the slip planes of dislocations which cause the material to deform. In other words, the fact of implanting nitrogen ions in the part to be treated makes it possible to increase the surface hardness of the part and thus make it very wear-resistant.

In addition, in application to aluminum alloy injection molds, since the nitrogen present in the aluminum is a base it has the effect of lowering the acidity that exists in the pits initiated by the chloride ions given off by the molded plastics. Thus, the corrosion associated with pit propagation is greatly reduced by the process of the invention.

Via the phenomenon of surface spraying caused by the passage of the incident ions, the process of the invention serves to erase the microroughnesses from the part, proportionately decreasing the development of pits, which generally take advantage of surface indentations to form.

As a result of these expedients, the process of the invention makes it possible to effectively treat regions of the part whose geometry is complex without thereby increasing either the duration of treatment or the risk of heating the part.

### BRIEF DESCRIPTION OF THE DRAWINGS

FIG. 1 represents a functional diagram of the device of the invention.

FIG. 2 represents examples of implantation distributions in an aluminum part, using an electron cyclotron resonance source producing N⁺, N₂⁺, and N₃⁺ ions and a single extraction voltage of 200 kV.

FIG. 3 represents the implantation profile obtained with a beam of N⁺ (3.3 mA), N₂⁺ (3.3 mA) and N₃⁺ (3.3 mA) and an extraction voltage of 200 kV, concentrated on an area of 1 cm² for 10 seconds. This profile represents the implanted nitrogen ion concentration (%) on the ordinate, as a function of the implantation depth expressed in angstroms.

FIG. 4 represents the ideal implantation profile of the same type as the preceding profile, obtained with a beam of N⁺(1.6 mA), N₂⁺ (3.2 mA), N₃⁺ (4.8 mA) and an extraction voltage of 200 kV, concentrated on an area of 1 crf² for 10 seconds.

### DETAILED DESCRIPTION OF EMBODIMENTS OF THE INVENTION

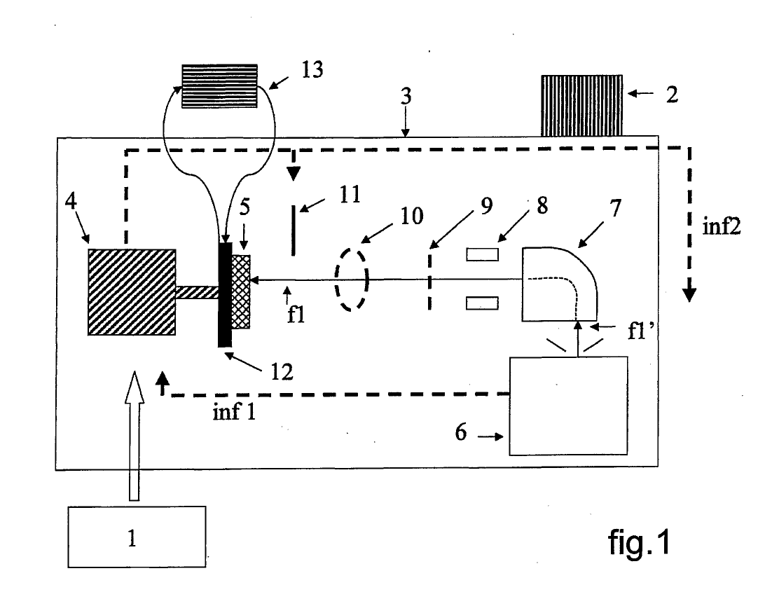

In FIG. 1, an apparatus according to the present invention is disposed in an enclosure 3 placed under vacuum by means of a vacuum pump 2. The purpose of the vacuum is to keep the beam from being intercepted by residual gases and to prevent the surface of the part from being contaminated with these gases during implantation.

This apparatus comprises an electron cyclotron resonance ion source 6, known as an ECR source. This ECR source 6 delivers an initial beam f1′ of multi-energy nitrogen ions, for a total current of about 10 mA (all charges combined, N⁺, N₂⁺, etc.) at an extraction voltage that is able to vary from 20 kV to 200 kV. The ECR source 6 emits the ion beam f1′ in the direction of first adjusting means 7-11, which are responsible for focusing and adjusting the initial beam f1′ emitted by ECR source 6 into an ion implantation beam fl that proceeds to strike a part to be treated 5.

These first adjusting means 7-11 comprise the following elements, from the ECR source 6 to the part 5:
a mass spectrometer 7 suitable for filtering the ions according to their charge and mass. This element is optional, since in the case where pure nitrogen gas (N₂) is injected, all the mono- and multi-charged nitrogen ions produced by the source can be recovered to yield a multi-energy nitrogen ion beam. Since the mass spectrometer is a very expensive element, the cost of the apparatus can be greatly reduced by using a beam of multi-energy ions obtained from bottled nitrogen;
lenses 8, whose purpose is to give the initial ion beam f1′ a chosen shape, for example cylindrical, with a chosen radius;
a profiler 9, whose purpose is to analyze the intensity of the beam in a perpendicular intersecting plane. This instrument of analysis becomes optional once the lenses 8 have undergone final adjustment at the time of the first implantation;
a current transformer 10, which continuously measures the intensity of the initial beam f1′ without intercepting it. The essential function of this instrument is to detect any interruption of the initial beam f1′ and make it possible to record variations in the intensity of the beam f1 during the treatment;
a shutter 11, which can be a Faraday cage, the purpose of which is to interrupt the trajectory of the ions at certain times, for example when the part is being displaced without being treated.

According to the preferred embodiment of the apparatus depicted in FIG. 1, the part 5 is movable with respect to the ECR source 6. Part 5 is mounted on a movable part holder 12 whose displacement is controlled by a numerical control machine 4, driven in turn by a postprocessor with calculations performed by a CADM (computer aided design and manufacturing) system 1.

The displacement of part 5 takes into account the radius of the beam f1, the outer and inner contours of the to-be-treated regions of the part 5, a constant rate of displacement, or a variable rate of displacement that depends on the angle of the beam f1 with respect to the surface and the number of passes already performed.

Control data (inf1) are transmitted from ECR source 8 to numerical control machine 4. These control data pertain to the state of the beam. In particular, ECR source 6 informs machine 4 when the ion beam f1 is ready to be sent. Other control data (inf2) are transmitted by machine 4 to shutter 11, to ECR source 6 and, where applicable, to one or more machines external to the apparatus. These control data can be the values of the radius of the ion beam, its emission rate and any other values known by machine 4.

In addition, part holder 12 is equipped with a cooling circuit 13 to evacuate the heat generated in part 5 during multi-energy ion implantation.

The operating procedure for the inventive apparatus is as follows:
the part to be treated 5 is fastened to part holder 12,
the enclosure 3 housing the apparatus is closed,
any cooling circuit 13 of part holder 12 is started,
the vacuum pump 2 is started in order to obtain a high vacuum in the enclosure 3,
once the vacuum conditions have been reached, ion beam f1′ is produced and adjusted via adjusting means 7-11,
when the beam is adjusted, the shutter 11 is raised and the numerical control machine 4 is activated and executes the displacement in terms of position and rate of the part 5 in front of the beam in one or more passes,
when the required number of passes has been reached, shutter 11 is lowered to cut off beam f1, the production of beam f1′ is stopped, the vacuum is broken by opening enclosure 3 to the ambient air, cooling circuit 13 (if any) is stopped, and treated part 5 is removed from enclosure 3.

There are two ways of reducing the temperature peak associated with the passage of the beam f1 over a given point on the part 5: increasing the radius of the beam (thus reducing the power per cm²) or increasing the rate of displacement.

If the part is too small to radiatively dissipate the heat associated with treatment, either the power of the beam f1 can be lowered (thereby increasing the treatment time), or the cooling circuit 13 housed in part holder 12 can be activated.

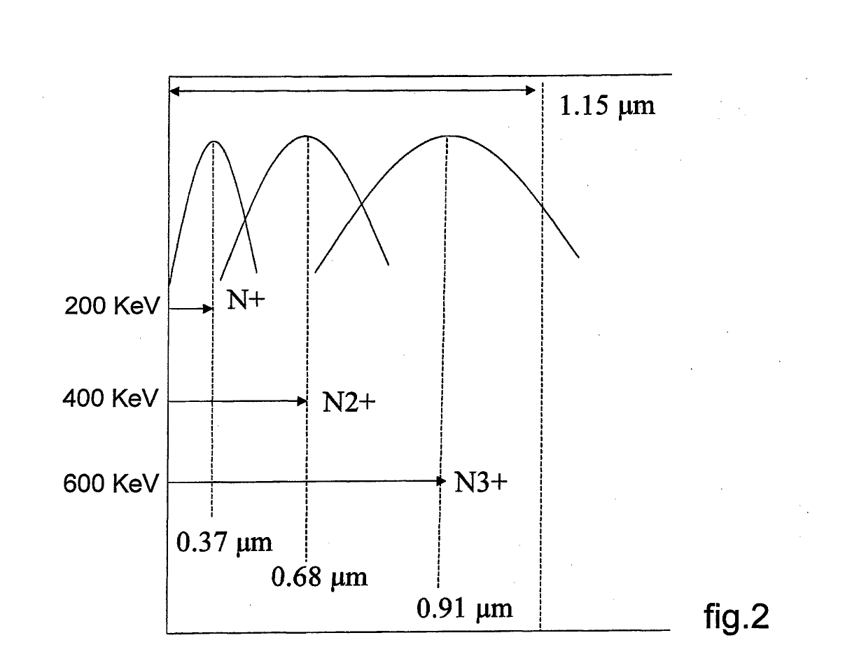

FIG. 2 represents an exemplary distribution of nitrogen ions N implanted in an aluminum part. In this example, the ion source delivers N⁺, N₂⁺ and N₃⁺ ions that are all extracted at one and the same extraction voltage, for example 200 kV. Thus, the N⁺ ions emitted by the ion source have an energy of 200 keV, the N₂⁺ ions have an energy of 400 keV and the N₃⁺ ions have an energy of 600 keV.

The N⁺ ions reach a depth of 0.37 μm±0.075 μm. The N₂⁺ ions reach a depth of about 0.68 μm±0.1 μm, and the N₃⁺ ions a depth of about 0.91 μm±0.15 μm. The maximum distance reached by ions in this example is 1.15 μm.

The distinctive feature of an ECR ion source 6 is that it delivers mono- and multi-charged ions, which makes it possible to implant multi-energy ions simultaneously at the same extraction voltage. In this way, a more or less well distributed implantation profile can be obtained simultaneously throughout the treated thickness.

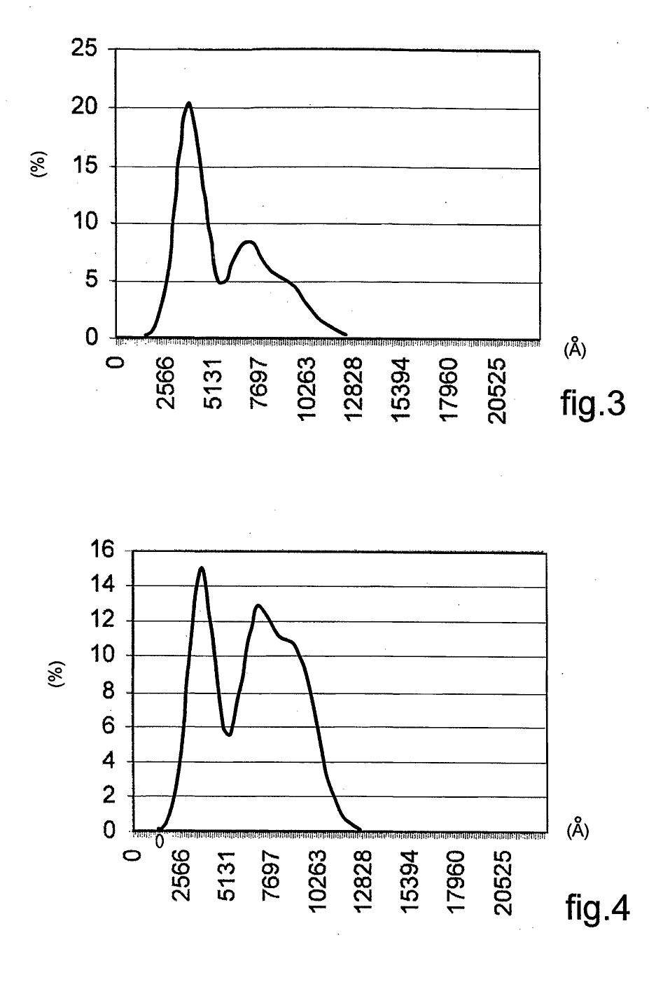

Considering for example an ECR source delivering a total current of 10 mA (3.3 mA for N⁺, 3.3 mA for N₂⁺, 3.3 mA for N₃⁺) with an extraction voltage of 200 kV, used to treat a 1 cm² aluminum part for about 10 seconds, the implantation profile is approximately that illustrated in FIG. 3. This profile reveals a concentration of:
20% N between 0.30 and 0.5 μm, corresponding to a 300% increase in hardness,
8% N between 0.5 and 0.85 μm, corresponding to a 200% increase in hardness, and
2% N between 0.85 and 1.1 μm, corresponding to a 35% increase in hardness.

The implantation profile is given an ideal distribution by adjusting the frequencies of the source 6 to produce an equidistributed distribution of the charge states of the ions from the source (same number of N, N₂⁺ and N₃⁺ ions per cm² and per second).

For instance, going back to the previous example, considering an ECR source delivering a total current of 10 mA (1.6 mA for N⁺, 3.2 mA for N₂⁺, 4.8 mA for N₃⁺) with an extraction voltage of 200 kV, used to treat a 1 cm² aluminum part for about 10 seconds, the implantation profile illustrated in FIG. 4 fluctuates between 6 and 14% over a thickness ranging from 0.25 μm to 1.1 μm.

With the same implanted ion concentration, the physical effect in terms of the hardness obtained by the simultaneous implantation of multi-energy ions is superior to that obtained by implanting mono-energy ions. This is because the dispersion of the aluminum nitride microcrystals due to the effectiveness of the mixing of the multi-energy ions (which are implanted at staggered depths) brings about an additional increase in hardness added to that obtained with a mono-energy ion beam.

1. An apparatus for implanting ions in an aluminum alloy element (5), comprising a source (6) for delivering ions accelerated by an extraction voltage, and a first adjusting means (7-11) for adjusting an initial beam (f1′) of ions emitted by said source (6) into an implantation beam (f1), wherein said source (6) is an electron cyclotron source producing multi-energy ions that are implanted in the element (5) at a temperature below 120° C. , the implantation of the multi-energy ions from the implantation beam (f1) being effected simultaneously at a depth controlled by the extraction voltage of the source.

2. The apparatus as in claim 1, wherein it further comprises a second adjusting means (1, 4, 12) for adjusting the relative positions of the element (5) and the ion source (6).

3. The apparatus as in claim 2, wherein the second adjusting means (1, 4, 12) comprises an element holder (12) movable so as to displace the element (5) during its treatment.

4. The apparatus as in claim 3, wherein the element holder (12) is equipped with cooling means (13) to evacuate the heat generated in the element (5) during the implantation of the multi-energy ions.

5. The apparatus as in claim 1 wherein, the first adjusting means (7-11) for adjusting the ion beam comprises a mass spectrometer (7) for sorting the ions produced by the source (6) according to their charge and mass.

6. The apparatus as in claim 1 wherein the adjusting means (7-11) for adjusting the initial ion beam (f1′) further comprises optical focusing means (8), a profiler (9), a current transformer (10) and a shutter (11).

7. The apparatus as in claim 1 wherein it is confined in an enclosure (3) equipped with a vacuum pump (2).

8. The apparatus as in claim 3, wherein the second adjusting means (1, 4, 12) for adjusting the relative positions of the element (5) and the ion source (6) comprises calculating means (1) for calculating said position on the basis of data related to the nature of the ion beam, the geometry of the part (5), the rate of displacement of the part holder (12) with respect to the source (6), and the number of passes already completed.

9. A process for treating an aluminum alloy by ion implantation employing an apparatus as in claim 1 wherein the multi-energy ion beam displaces relatively with respect to the element (5) at a constant rate.

10. A process for treating an aluminum alloy by ion implantation employing an apparatus as in claim 1, wherein the multi-energy ion beam displaces relatively with respect to the element (5) at a variable rate that takes into account the angle of incidence of the multi-energy ion beam with respect to the surface of the element (5).

11. The process of claim 9 wherein the multi-energy ion beam is emitted at a constant emission rate and constant emission energies.

12. The process of claim 9 wherein the multi-energy ion beam is emitted at a variable emission energies controlled by the ion source (6).
